(19) United States
(12) Patent Application Publication
(10) Pub. No.: US 2009/0011052 A1
(43) Pub. Date: Jan. 8, 2009

(54) Anti-HIV medicinal herbs composition, preparation thereof and use of the same

(76) Inventors: Zelin Li, Beijing (CN); Yue Zeng, Beijing (CN); Yi Zeng, Beijing (CN); Yan Tsang, Beijing (CN)

Correspondence Address: SOFER & HAROUN LLP., 317 MADISON AVENUE, SUITE 910, NEW YORK, NY, 10017, US

(21) Appl. No.: 11/715,302

(22) Filed: Mar. 7, 2007

(30) Foreign Application Priority Data
Sep. 30, 2004 (CN) CN200410080516.8
Sep. 27, 2005 (CN) PCT/CN2005/001579

(51) Int. Cl. A61K 36/258; A61P 31/18

(52) U.S. Cl. 424/728

(57) ABSTRACT

A medicinal herbs composition includes the extract powders of Scutellaria baicalensis Georgi and Rubia cordifolia L, and powders of ginsenoside and Cordyceps sinensis (Berk.) Sacc. The present invention further concerns the preparation method of the said medicinal herbs composition and the use of the same for treating AIDS.

### FIELD OF THE INVENTION

The present invention concerns a medicinal herbs composition containing the extract powders of Scutellaria baicalensis Georgi and Rubia cordifolia L, and powders of ginsenoside and Cordyceps sinensis (Berk.) Sacc. The present invention further concerns the preparation method of the said medicinal herbs composition and the application of the said drug to treat AIDS.

### BACKGROUND OF THE INVENTION

As one of the drugs for resolving heat and dampness, Scutellaria baicalensis Georgi is the root of Lamiaceae plants, that mainly containing flavoids. Its major pharmaceutical effects include anti-inflammation, anti-allergy, antimicrobial, antipyretic function and detoxification. Also known as rubia root, hemorrhage stopper and blood-activating grass, Rubia cordifolia L is the dry root and bulb of Rubiaceae plants. The main ingredients of rubia are liposoluble anthraquinones, reduced anthraquinones and their glycosides and water-soluble cyclohexapeptides. Additionally it is also rich in calcium ions. Its major pharmaceutical effects include blood-cooling, hemostasis, stasis-removing, circulation-boosting, tumor-killing and antimicrobial. With sweet and slightly bitter tastes plus a mild action, the gingsen roots are capable of adjusting the energy flows, nurturing the blood, quieting the spirits, sharpening the wisdom, generating the saliva, relieving the cough, replenishing the nutrients and strenghthening the body. Known as “the King of Herbs”, it has been the preferable choice of body-regulating drug ever since the ancient times in China. And the cordyceps are cousins of Cordyceps sinensis (Berk.) Sacc and belong to the ascomycetes hypocreaceae. The they are made up of young insect bodies of Hepialus armoricanus oberthur of Vespertilionidae hosts. The cordyceps contain crude proteins, lipids, crude fibers, carbohydrates, ashes, cordycepic acid, cordycepin, cordyceps polysaccharides, nucleotides, peptides and eicosane. The major pharmaceutical effects include spirit-lifting, yang-boosting, resistance-enhancing, energy-nurturing, kidney-nourishing, cough-relieving, body-stengthening and longevity-adding. Also it is also capable of anti-inflammation, antiarrhythmic and tumor-killing.

As demonstrated by many experiments, ginseng can enhance the physical and intellectual activities of animals and human and boost the body's non-specific resistance against a variety of noxious stimulations. Within the therapeutic range, it has no interference with the normal physiological functions and no side effects. It is considered as a class of beneficial and harmless strengtheners and tonics for the whole body.

Building upon the discovery of the said pharmacological effects of ginseng, the researchers have conducted researches of the ginseng extracts. It is found that more than 30 kinds of ginsenosides exist with major pharmacological actions, such as Rb1, Rb2, Rb3, Rc, Rd, Re, Rg, Rh1, Rh2, F2, pseudoginsenoside F1, RTs and American Ginsenoside. The main pharmacological studies of these ginsenosides include the effects of anti-aging, immunity-boosting and lipid-lowering, and some changes of heart and blood vessels. But up until now, there is still no report of employing ginseng, ginseng extracts or any ginsenoside to treat AIDS. AIDS stands for Acquired Immunodeficiency Syndrome. The causative agent is Human Immunodeficiency Virus. It mainly attacks the human immune system, especially the CD4 lymphocyte. At last, the body's immune functions are destroyed and the resulting opportunistic infections cause the patient's death.

Suramin was the first reported drug to fight against the HIV virus. In 1985, AZT was found to possess the in vitro anti-HIV activity. In 1986, the clinical trial was carried out. In 1987, AZT became the first drug approved by FDA for treating AIDS. But the main issues were its drug toxicities and resistance. Other drugs appeared in the subsequent years. Up until now, more than 20 anti-HIV drugs have been cleared for uses in the US. According to their mechanisms of action, they fall mainly into 3 categories. Except for T20—a cellular entry-blocker approved at the year-end of 2002, all the other drugs belong to the viral reverse transcriptase (RT) inhibitors, such as AZT, DDC, DDI and viral protease inhibitors. FDA had already approved five drugs of protease inhibitors, namely saquinavir, ritonavir, indinavir sulfate and nefinavir, etc. In 1995, the American scientists adopted the “Triple Combination” regimen of two RT inhibitors and one protease inhibitor. Known as HAART, such therapy is currently in common uses. This therapy has enhanced the treatment outcome and further prolonged the patient's life. It has been now used for 6-7 years. And a small number of patients still survive.

There are currently three drugs available for the cocktail of HAART in China. But there are only RT inhibiting drugs and the protease inhibitors are still lacking. And the toxicities are so serious that nearly 20% of the patients cannot tolerate. Therefore there exist the issues of drug toxicities and resistance in the clinical applications. As stated above, T20 is able to block the cellular entry of virus. However, T20 cannot be taken orally since it is a peptide. It must be injected. And the price is quite expensive. Therefore it is imperative to develop anti-HIV drugs with minor toxicities.

The traditional Chinese medicine is such a great trove of treasures that it is quite worth our efforts to explore and carry forward. Through more than ten years' researches, the inventor has found out that some herbal extracts, ingredients or mono-components have well-defined anti-HIV activities. The targets of their anti-HIV activities were studied. With such advantages as a cheaper price and smaller toxicities over HAART, they can markedly boost the immune functions. And it is necessary to constantly update the therapeutic cocktail since HAART has the problem of drug resistance. So there are broad prospects of applying TCM to treat AIDS in clinical settings.

### SUMMARY OF THE INVENTION

The present inventor has found out that the medicinal herbs composition consisting of extract powders of Scutellaria baicalensis Georgi and Rubia cordifolia L, and powders of ginsenoside and Cordyceps sinensis (Berk.) Sacc has marked effects of treating AIDS.

Therefore, on one hand, the present invention concerns a medicinal composition consisting of 10-30 weight parts of extract powders of Scutellaria baicalensis Georgi and 10-25 weight parts of Rubia cordifolia L, and 18-25 weight parts of powders of ginsenoside and 30-55 weight parts of powders of Cordyceps sinensis (Berk.) Sacc. based on 100 weight parts of the composition.

Within the said composition, more preferably, the said extract powders of Scutellaria baicalensis Georgi is in amount of 18 weight part, and Rubia cordifolia L, is in amount of 17 weight parts and powders of ginsenoside is in amount of 18 weight parts and Cordyceps sinensis (Berk.) Sacc is in amount of 47 weight parts respectively.

1. Within the said composition, the extract powder is ethanol extract powder and can be harvested from any known technology in the art. The extract powders of Scutellaria baicalensis Georgi as well as Rubia cordifolia L can be prepared preferably as given below. And ginsenoside powder may be a market-sold product meeting the quality standard for preparation. The ginsenoside powder as optimal selection standard contains 15-20 wt % Rb1, 15-20 wt % Rb2, 30-90 wt % Rb3 and 30-90 wt % Rc. The said powder of Cordyceps sinensis (Berk.) Sacc is a powder of natural Cordyceps sinensis (Berk.) Sacc or artificially cultivated Cordyceps sinensis (Berk.) Sacc.

On the other hand, the present invention concerns a preparation method of the said medicinal herbs composition, comprising the steps of:

a) Crushing the crude herb of Scutellaria baicalensis Georgi as raw material medicine, adding 4-5 folds of ethanol for immersing for at least 6 hours, pouring the immersed Scutellaria baicalensis Georgi and immersion solution together into an osmotic filtration tank for filtration in rate at 1-4.5 ml/kg.minute⁻¹; then adding ethanol and osmotic-filtrating simultaneously until making the collected osmotic filtration fluid equal in weight to 10-20 folds of the crude herbs; collecting and concentrating the osmotic filtration fluid to form final concentrated solution having weight ratio of water and concentrated fluid at 1:1.1-1.35; spray drying the concentrated fluid into extract powder of Scutellaria baicalensis Georgi.;

b) Simultaneously or sequentially crushing the crude herb of Rubia cordifolia L, adding 4-5 folds of ethanol for immersing for at least 6 hours, pouring the immersed Rubia cordifolia L, and immersion solution together into an osmotic filtration tank for filtration in rate at 1-4.5 ml/kg.minute⁻¹, then adding ethanol and osmotic-filtrating simultaneously until making the collected osmotic filtration fluid equal in weight to 10-20 folds of the crude herbs; collecting and concentrating the osmotic filtration fluid to form final concentrated fluid having weight ratio of water and concentrated fluid at 1:1.1-1.35; spray drying the concentrated fluid into extract powder of Rubia cordifolia L.;

c) mixing 10-30 weight parts of extract powder of Scutellaria baicalensis Georgi, 10-25 weight parts of extract powder of Rubia cordifolia L, 18-25 weight parts of ginsenoside powder and 30-55 weight parts of powder of Cordyceps sinensis (Berk) Sacc to obtain the medicinal herbs composition based on 100 weight parts of the composition.

Within the above method, preferably 30-70%, more preferably 50% ethanol is used.

Additionally the present invention concerns use of the said medicinal herbs composition in preparation of anti-HIV drugs.

The said medicinal herbs composition may be used in combination with the ordinary anti-HIV drugs. The said ordinary anti-HIV drugs include such as AZT, DDC, DDI, saquinavir, ritonavir, indinavir sulfate and nefinavir.

The dosing formulations of the said anti-HIV drugs may be liquids or solids. For example, the liquid formulations can be true solution types, colloid types, microgranular forms, emulsoid forms and suspensive forms. Other formulations include tablets, capsules, drops, aerosols, pills, pellets, solutions, suspensions, emulsions, granules, suppositories and freeze-dry powder injections and the like.

The medicinal herbs composition of the present invention may be made into common preparations, sustained-release preparations, controlled-release preparations, targeted preparations and varieties of mircosomal drug delivery systems.

All carriers known in the art can be used so as to prepare the unit dosage formulations into the tablets, e.g. carriers as dilutes and absorbents including starch, dextran, calcium sulfate, lactose, mannitose, sucrose, sodium chloride, glucose, urea, calcium carbonate, white clay, microcrystal cellulose and aluminum silicate, etc; lubricant and adhesive, such as water, glycerol, polyethylene glycol, ethanol, propanol, starch slurry, dextran, syrup, honey, glucose solution, Arabian glue, gelatin glue, carboxylmethylcellulose sodium, sdshellac, methylcellulose, potassium phosphate and PVP, etc; disintegrant, such as dry starch, alginate, agrose powder, algin starch, sodium bicarbonate, citrate, calcium carbonate, polyoxyethylene sorbitan alphate, sodium dodecylsulfonate, methylcellulose and ethylcellulose, etc; disintegration inhibitor, such as sucrose, glycol tristearate, cocoa butter and hydrogenated oil, etc; absorption promoter, such as quaternary ammonium and sodium dodecylsulfate, etc; lubricant, such as talcum powder, silicon oxide, corn starch, stearate, borate, liquid paraffin and polyethylene glycol, etc. Other carriers, such as polyacrylic acid resins and liposome; water-soluble carriers, such as PEG4000, PEG6000 and PVP, etc. Also the tablets may be further prepared into coated pills, such as sugar-coated, thin membrane-coated, enteric coated, or double-layered and multi-layered tablets.

For example, it is feasible to adopt extensively all the known carriers in the art so as to prepare the unit dosage formulations into the pellets. Examples of carriers are dilutes and absorbents, such as glucose, lactose, starch, cocoa butter, hydrogenated plant oil, PVP, kaolin and talcum powder, etc; adhesive, such as Arabian glue, tragacanth gum, gelatin, ethanol, honey, liquid sugar and rice or flour paste, etc.; disintegrant, such as agrose powder, dry starch, alginate, sodium dodecylsulfonate, methylcellulose and ethylcellulose, etc.

For example, with the purpose of preparing the unit medicine into the capsules, the active ingredients of the invented medicinal composition can be mixed with various said carriers to obtain a mixture. And the mixture is placed into hard gelatin or soft capsules.

For example, the herbs composition be prepared to be injections in form, such as solutions, suspensions, emulsions and freeze-dry powder injections. This kind of preparation can contain water or no water, may contain one and/or multiple pharmacologically acceptable carriers, dilutes, adhesives, lubricants, preservatives, surfactants or dispersers. For dilutes, water, ethanol, polyethylene glycol, 1, 3-propylene glycol, ethoxylated prisorine, polyoxylated prisorine and polyoxyethylene sorbitan alphate are included. In addition, it is feasible to add appropriate amounts of sodium chloride, glucose or glycerol into the injections to prepare the isotonic injections. Furthermore the conventional solubilization boosters, buffering agents and pH adjusting agents may be added. These supplemental materials are commonly used in this field.

Besides, if necessary, the colorants, preservatives, spices, taste modifiers, sweeteners and other substances may be also included in the present medicinal herbs composition.

For the purposes of achieving the medication goals and enhancing the therapeutic effects, this medicinal herbs composition may be administered by any well-known dosing method, preferably by oral intake.

The dosages of the medicinal herbs composition are determined by many factors, such as the severity of disease courses for the AIDS patients, sex, age, body weight, disposition, individual response, dosing routes, dosing frequency and treatment goals. As a result, the therapeutic dose range of this invention is large. Generally speaking, the practical doses of drug ingredients of this invention are well-known among the professionals in this field. It is possible to make adequate adjustments to the actual drug quantity of the final preparation of the medicinal herbs composition so as to achieve the effective therapeutic levels and accomplish the preventive or therapeutic goals of this invention. The daily proper dose range of the medicinal herbs composition is 0.03-0.50 mg/Kg body weight. The above dose may be administered in 2, 3 or 4 times per day. The administration is subject to the clinical experiences of physicians and influenced by the dosing plans through other therapeutic approaches.

### BRIEF DESCRIPTION OF DRAWINGS

FIG. 1 shows the dynamic changes of CD₄+ lymphocytes within ZL-1-treated monkeys.

FIG. 2 shows the inhibiting effects of the medicinal herbs composition upon the viral replication of SIV-infected monkeys.

FIG. 3 shows the studies of the acting targets for rubia (CD), ginsenoside (JH) and the present medicinal herbs composition (ZL-1).

FIG. 4 shows the effects of the medicinal herbs composition upon the co-receptors of CXCR4 and CCR5.

FIG. 5 indicates the testing results of effects of JHR (one of the ingredients of the medicinal herbs composition) upon the CD₄ receptor.

FIG. 6 shows the conjugation effect of rubia (CD) and gp₄₁ (transmembrane protein.

FIG. 7 shows the conjugation effect of ginsenoside (JHR) to gp₄₁.

FIG. 8 shows the changes of CD₄ of a patient receiving ZL-1 treatment for 6 months.

### DETAILED DESCRIPTION OF THE INVENTION

The present invention is described in combination with the figures in details.

### Example 1

### Preparation of this Invented Medicinal Herbs Composition

180 kg Scutellaria baicalensis Georgi and 150 kg crude rubua herb as raw material medicine were taken and crushed, and 1400 L of 50% ethanol is added for immersing for 6 hours. The said raw material medicine and immersion solution together were poured into an osmotic filtration tank. The rate of osmotic filtration was at 3-4.5 ml/kg.minute⁻¹. The same ethanol was added in batches and continued to make the osmotic filtration. Then 4000 L of osmotic filtration fluid was collected. Through recycling concentrated ethanol osmotic filtration fluid, the final concentrated fluid with the weight ratio of water:concentrated fluid at 1:1.1-1.35 was harvested. The above concentrated fluid was dried by spray technology into powder. The harvest rate of extract powders of Scutellaria baicalensis Georgi and Rubia cordifolia L. were 8-10% and 10-15% respectively. So the obtained dry powder was equivalent to 18 kg extract powder of Scutellaria baicalensis Georgi and 17 kg extract powder of Rubia cordifolia L. the harvested dry powders were mixed with 18 kg ginsenoside powder and 47 kg Cordyceps sinensis (Berk.) Sacc powder to obtain the invented medicinal herbs composition ZL-1. Using the process well-known by the person skilled in the art, appropriate amounts of medicinal carriers were used to form the capsule powder required by this invention. Then the capsules were packaged for later usage. Each capsule contains around 0.5 g of the said medicinal herbs composition.

### Example 2

180 kg crude herb of Scutellaria baicalensis Georgi as raw material medicine was taken and crushed, following by adding 720 L of 50% ethanol for immersing for 6 hours. The immersed raw material medicine and immersion solution together were poured into an osmotic filtration tank. The rate of osmotic filtration was at 3-4.5 ml/kg.minute⁻¹. Then 50% ethanol intermittently was added and continued to make the osmotic filtration. 2200 L of osmotic filtration fluid was collected. Through evaporating the concentrated ethanol osmotic filtration fluid, the final concentrated fluid was harvested with the weight ratio of water:concentrated fluid at 1:1.1-1.35. The above concentrated fluid was dried by spray into powder and obtaining 18 kg extract powder of Scutellaria baicalensis Georgi.

150 kg crude herb of Rubia cordifolia L. as immersed raw material medicine was taken and crushed and following by adding 600 L of 40% ethanol for immersing for about 6 hours. The immersed raw material medicine and immersion solution together were poured into an osmotic filtration tank. The rate of osmotic filtration was set up at 3-4.5 ml/kg.minute⁻¹. Then 40% ethanol was intermittently added and continued to make the osmotic filtration. 1800 L osmotic filtration fluid was obtained. Through evaporating the concentrated ethanol osmotic filtration fluid, the final concentrated fluid was harvested with the weight ratio of water:concentrated fluid at 1:1.1-1.35. The above concentrated fluid was dried by spray into powder and obtaining 17 kg extract powder of Rubia cordifolia L.

The harvested dry powder of Rubia cordifolia L. was mixed with 18 kg ginsenoside and 47 kg Cordyceps sinensis (Berk.) Sacc to obtain the medicinal herbs composition ZL-1 and packaged into the capsules for later usage.

### Experiment 1

### Pharmacodynamic Studies of the Medicinal Herbs Composition of the Present Invention

In Vitro Experiment

Three types of cells (such as MT4, Hela-CD₄, PBMC) were infected respectively with HIV-1 virus for observed the inhibiting effects of ZL-1, as prepared within Example 1, upon the HIV-1 virus replication.

(1) MT4 Cell

Viral strains: HIV-1 and NL4 Cell Line

Method: The medicinal herbs composition obtained in Example 1 was prepared into the concentration of 1 mg/ml as drug solution and diluted into different concentrations for later uses during the experiment. \textcircled{1}: The experiment was carried out on a 96-well culture plate, 100 μl drug solution was added into each well and making each concentration's drug solution at least duplicate. Freshly cultured MT4 cells were additionally fetched in tubes of 5×10⁵ cells. HIV-1 virus 1×10³TCID₅₀/ML, 37° C. was added into the cells that were further cultured in a CO₂ incubator. The culture after 2 hours was centrifuge and the supernatant was discarded and washed once with RPMI1640. The free virus was removed and added to the culture medium at 5×10⁵/1 ml. 100 μL HLV-1-infected cells were added into all the drug wells of a 96-well plate (5×10⁴/100 μL) for culturing at 37° C. within a CO₂ incubator. On the third day, 100 μL supernatant was sucked out from each well replaced by 100 μL of fresh drug solution with the same concentrations as the wells. 100 μL of culture medium was added as a control group. On the sixth day, the supernatant was taken from each well. The Microelisa method and reagents were adopted to measure the amount of P₂₄ antigen. For each experiment, the virus control (VC), cell control and AZT positive drug control were used. Based upon the amount of P₂₄ antigen (P₂₄-Ag), the inhibition rate was calculated (IR) according to the following formula

$$IR = \frac{VC well {P}_{24} \text{-} Ag - Drug group {P}_{24} \text{-} Ag}{VC well {P}_{24} \text{-} Ag} \times 100$$

Different IRs were obtained for drug solutions of different concentrations. After statistic processing, IC₅₀ was obtained.

(2) Hela-CD₄ Cell

Virus: Single-life-cycle reporter HIV virus was acquired by transfection with HIV plasmids.

Method: Hela-CD₄-LTR-gal cell was innoculated into a 24-well plate for standing for 24 hours and let it absorb and adhere to the wall. On the second day, the supernatants were sucked away from the wells and 100 μl drug (drug control) or drug and HIV-1 (drug solutions with different concentrations) or culture medium (Mock) were added. After 2 hours, 200 μl of identical drug solutions or culture medium was added into each well for incubating at 37° C. in a CO₂ incubator for 48 hours and detecting by the following method.

Fixation: the supernatants were sucked away from each well and adding the fixative solution (1 ml), and then by staining with K₄[Fe(CN)₆].3H₂O, K₃[Fe(CN)₆] and X-gel.

Counting: For the blue cell counts (BCC) within each well, the following formula is used to calculate the IR and then IC₅₀.

$$IR = \frac{Mock well B C C … Drug well B C C}{Mock well B C C} \times 100$$

(3) PBMC Cell

Virus: NL4-3

Method: the freshly harvested PBMC (human peripheral blood newly isolated lymphocytes) was collected, following by counting and centrifuging at 1200 rpm, discarding the supernatant and preparing the solution of 3×10⁶ cell/ml. The culture medium was pre-treated with IL2. (1 μl 1000×IL2 for every ml culture medium) at 37° C. overnight. For the experiment, 5×10⁶ was counted as one infection unit. The duplicate series of JHR at the same concentration of 0.4 mg/ml, virus control and cell control were set up. On a 24-well plate, each well contained 5×10⁶ cells in drug solutions or culture medium. NL4.3 virus (HIV-1) with the viral load of 4×10⁴ IU in each well was blown and shaken thoroughly and transferred into a 12-well plate. Then 1.5 ml of the identical drug solution or culture medium were added to make a total volume of 2 ml in order to incubate at 37° C. The supernatant was taken every 3-4 days in 100 μl Fetched for each well and stored at −80° C. When finishing collecting, RT was measured and the drug group was compared with virus control group to calculate the inhibition rate.

The results were given as below:

MT4 cell strain: IC50=139 μg/ml.

Hela-CD cell strain: IC50=54.8 μg/ml.

PBMC cell (one of components of the medicinal herbs compositions—JHR): (human peripheral blood newly isolated lymphocytes), IC50=77 μg/ml on Day 6 of medication.

The results showed that ZL-1 had marked in vitro inhibiting effects upon HIV-1.

Experiment 2

In Vivo Experiment of this Medicinal Herbs Composition—Therapeutical Effects upon Rhesus monkey

Experiment animals: 5 females and 5 males Rhesus monkey as experimental animal with 4.5-5.5 Kg/each for a total of 10 were used, purchased from Primate Breeding Center, Research Institute of Experimental Animals, Chinese Academy of Medical Sciences. They were fed with commercial popped feeds. No abnormal physical examination was found before the experiment. Potential infections with SIV, SRV Type D and STLV-I were excluded by the serum IFA antibody test.

Infection strain and dosage: The SIVmac251 strain was donated by Dr. Darx of Aaron Diamond AIDS Research Center of the US. Each animal was intravenously infected with 1 ml virus solution equivalent to 3MID₁₀₀ viral load.

Medication and treatment protocol: ZL-1 and AZT were supplied by the present inventors. The formulation was capsuled and the animals were divided into AZT positive drug treatment group and SIVmac251 infection control group. Within each group, there were 3-4 animals with equal females and males. 10 days before the viral infection, ZL-1 treatment group animals were dosed orally with 2 capsules qd for 70 days continuously; Starting from 30 minutes post-infection, AZT positive drug control group animals were dosed orally with 100 mg once daily for 60 days continuously. No drug treatment was offered for SIVmac₂₅₁ infection control group.

Observation pre- and post-infection: Besides other pre-infection physical examinations, body weight and blood-drawing were made for each monkey (conducting all parameter tests). Continue performing general observations and tests were also done during and after the treatment. At the pre-determined timepoints, blood was drawn for the monkey to measure the plasma viral titer, blood CD₄⁺ count and antibody titre. After finishing the 60-days experiment, the animals were killed.

Plasma virus isolation: After the animals were infected with SIVmac₂₅₁, to collect the blood samples were collected respectively at Days 7, 11, 14, 21, 28, 42, 60 and the plasma were separated by heparin anti-clotting. Beginning from 200 μl, the plasma was serially diluted in 5-fold increments into a 24-well plate. The volume of 1 ml within each well was reached by adding 10% bovine serum RPMI-1640. Then 0.6 ml CEMX₁₇₄ cell solution with 10⁵ SIVmac₂₅₁(sensitive strain) was dispensed for further standing in a 37° C. CO₂ incubator in order to observe and propagate the cell line every 3-4 days. The viral titre was chosen for the sample with the lowest inoculation quantity but showing the typical patterns of cellular confluence. For each sample, a total of 8 titres, that is 200 μl, 40 μl, 8 μl, 1.6 μl, 0.32 μl, 0.064 μl, 0.0128 μl and 0.00256 μl. Each was equivalent to 5, 25, 125, 625, 3125, 15625, 78125 and 390625 TCID values respectively of each microliter of plasma SIV virus. After observing the samples for 3 weeks, the viral isolation results were judged.

Measurement of blood CD₄⁺ count: Blood samples were drawn from the animals before infection and 14, 28, 42 and 60 days after infection with SIVmac₂₅₁ Ficoll lymphocyte separation fluid was adopted to isolate the peripheral lymphocytes. After tagging the anti-CD4 monoclonal antibody with direct fluorescence, the cells were counted on FACS machine to obtain the CD₄⁺ percentage. In combination with WBC count with DC of routine blood examination, the total count of blood CD₄⁺ lymphocytes was calculated.
1. Dynamic changes of CD₄+ lymphocytes within the Rhesus monkey before and after drug treatment

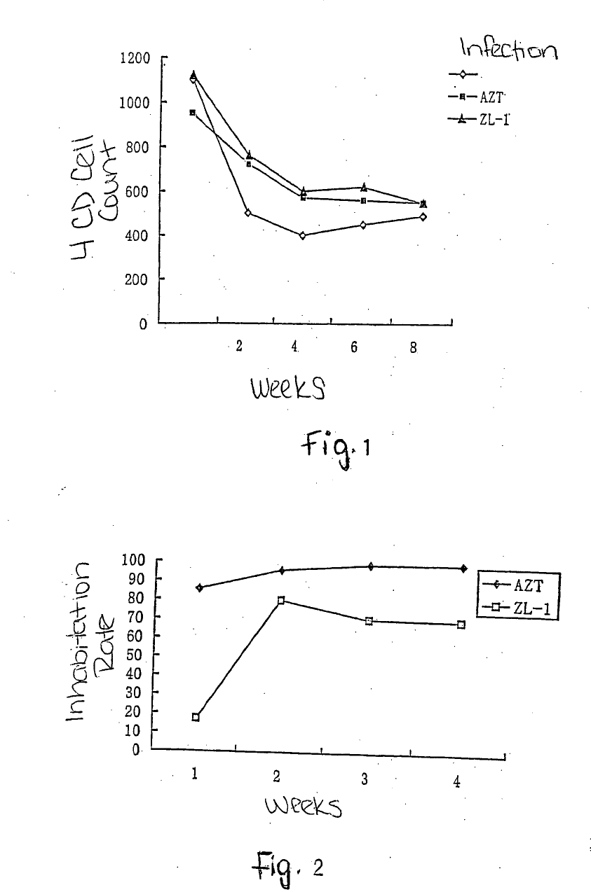

Results: As shown in FIG. 1 and Table 1.

TABLE 1

| | | | Difference | | Difference | | Difference | | Difference |
|---|---|---|---|---|---|---|---|---|---|
| | | 2 week | with | | with | | with | | with |
| Groups | Pre-experiment | infection | control | 4 week | control | 6 week | control | 8 w | control |
| Infection | 1069.66 | 527.30 | | 402.61 | | 460.07 | | 494.72 | |
| | | | | | | | | (371.04) | |
| ZL-1 | 1096.86 | 762.74 | 235.44 | 594.57 | 191.96 | 608.69 | 148.62 | 514.37 | 143.33 |
| | | | (44.65%) | | (47.68%) | | (32.3%) | | (38.63%) |
| AZT | 959.86 | 714.51 | 187.21 | 540.57 | 137.96 | 543.31 | 83.24 | 530.95 | 159.9 |
| | | | (35.5%) | | (34.27%) | | (18.09%) | | (43.0%) |

The above results demonstrated that, after the monkey was infected with SIV, the viral load was rising while the CD⁴ cell count dropped sharply. However, within the ZL-1 treatment group 2 and 4 weeks post-infection, 45-48% cells were spared of the viral attacks while 35% cells within the AZT group received protection. Even the 6 and 8 weeks post-infection, 32-38% cells within the ZL-1 treatment group were still protected.
2. Viral load changes within the Rhesus monkey before and after medication

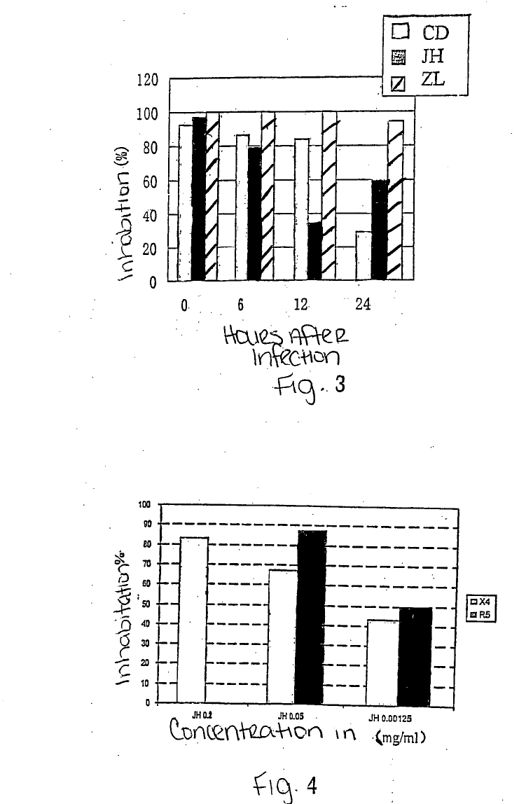

The results were shown as below and in FIG. 2.

Rhesus monkey treatment experiment (acute infection of Rhesus monkey when SIVmac₂₅₁)

Post-medication weeks

| | 1 | 2 | 3 | 4 |
|---|---|---|---|---|
| Inhibition | 17.0% | 80.2% | 70.7% | 70.2% |
| rate (%) | | | | |

Results demonstrated that the medicinal herbs composition had marked inhibiting effects upon the SIV infection in Rhesus monkey.

The traditional agent of treating AIDS, that is AZT, has marked inhibiting effects upon SIV with an inhibition rate over 90%. The said medicinal herbs composition has also marked inhibiting effects. Within the dosing range, the inhibition rate can be maintained at 70-80%.

### Example 3

### Action Targets of the Medicinal Herbs Composition of the Present Invention

1. Detection of Action Phases During the Viral Life Cycle

Object: It is to observe which phase(s) of viral life cycle are targeted by the said medicinal herbs composition, including viral entry into cells, reverse transcriptase, integrase, transcription and proteases. The viral “single life cycle” model was employed to study the target of drug actions.

(1) MAGI method: The recombinant virus has the LTR of HIV. The reporter gene of β-galactosidase is expressed to form one kind of viral “single life cycle” model. As mentioned as above, this model employs K₄[Fe(CN)₆], K₃[Fe(CN)₆] and x-gel to stain the Hela-CD₄ cell. The blue cells under the microscope denote the presence of viral genes.

(2) Luciferase Method

The recombinant and transfected VSVG virus and the cell line of H9 strain were adopted. The examination method was to detect the activity of Luciferase by illumination. A heavier viral load denoted a higher enzymatic activity.

Experiment method: After viral infections, the cells were divided into different groups. They were also dosed respectively at 0, 6, 12, 18, 24 and 36 hours after infections. At 48 hours post-infection, the method of MAGI or Luciferase was adopted.

The above two testing methods were both viral models of “single life cycle”. Their major advantages showed that different infection times denoted different phases of viral replications. At Hour 2-6, the virus was entering the cell. At Hour 10-14, it was the phase of reverse transcription. After Hour 20, it was the phase of recombination and transcription. As a result, dosing at different timepoints acted upon specific target points. This invention experiment had analyzed different target points of JHR and Rb3.

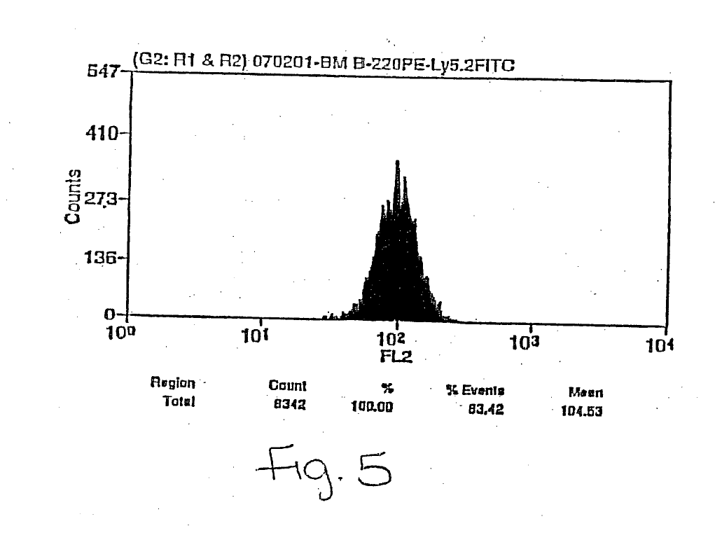

As shown in FIG. 3, the results demonstrated that ZL-1 blocks the viral entry into the cells and acts upon the phase of reverse transcriptase and integrase. The rubia extract (CD) within the herbs composition ZL-1 is only active during the phases of viral entry and reverse transcriptase. And ginsenoside (JHR) is effective during the phase of viral entry and integrase so that the herbs composition is superior to any single extract.

2 Detection of Co-Receptor CXCR4CCR5

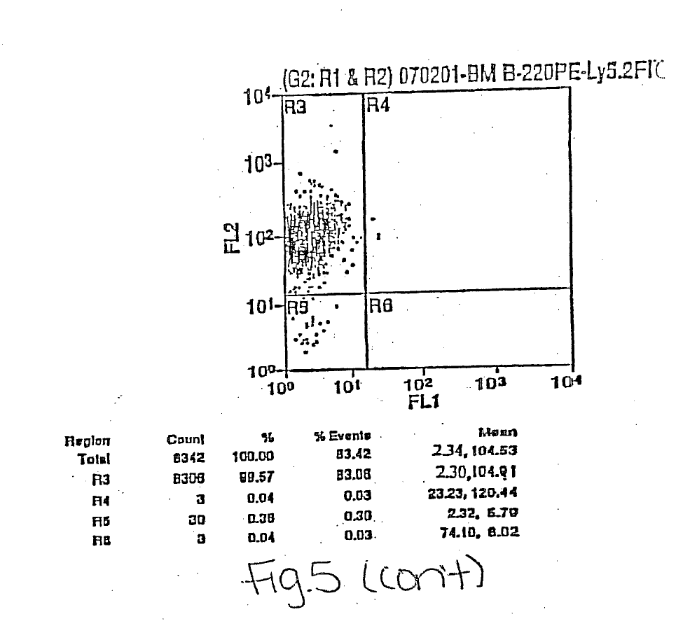

For the experiment, the MAGI method was adopted: method was used with the same as above to detect CXCR4 receptor by T lymphotropic lymphocytes as receptor, and virus was NL₄. Macrophage-tropic lymphocytes was used to detect CCR5 receptor, and virus YU2 was used as virus. The results were shown in FIG. 4.

As seen from FIG. 4, the medicinal herbs composition had both inhibiting effects upon the co-receptors CXCR4 and CCR5. So it could be demonstrated that the effects were probably acting upon the shared CD₄ receptors of T lymphotropic and macrophage-tropic lymphocytes or during the virus-cell fusion. These two co-receptors were not targeted.

3. Detection of Actions Upon CD₄ Receptor

JHR—one of ingredients of the medicinal herbs composition was used in the present study used.

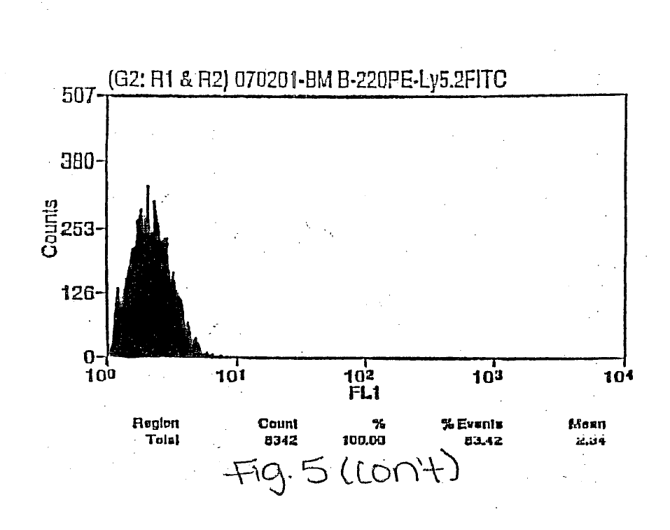

The method of flow cytometry was used for measurement. Method was given as follows: SupT1 cell with drug was co-incubated at 37° C. for 2 hours and washed with PBS+2% FCS. In a 4° C. ice bath, CD₄PE was added and sit for 30 minutes. After further washing and centrifuging, CD₄ monoclonal Ab was added to, followed by incubating in ice bath for 30 minutes; again after washed and centrifuged and sit in ice bath. The cells were suspended in 50 μl secondary Ab anti-mice-FITC for 20 minutes, followed by washing once and were suspended in 300-500 ul PBS/2% CS+PI. FACS testing was performed. The testing results were shown in FIG. 5.

Results showed that JHR had no effects upon CD₄ receptor.

4. Detection of Viral Transmembrane Protein gp41

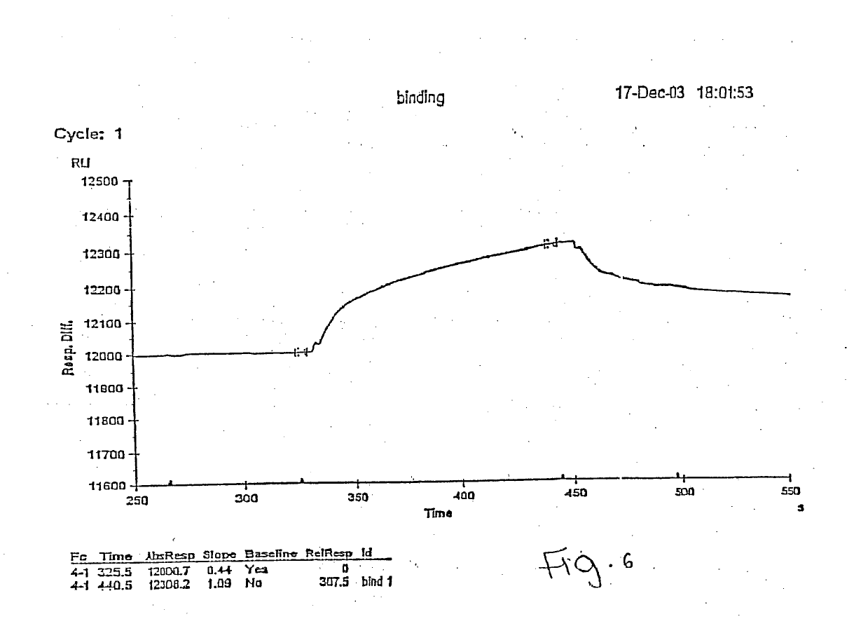

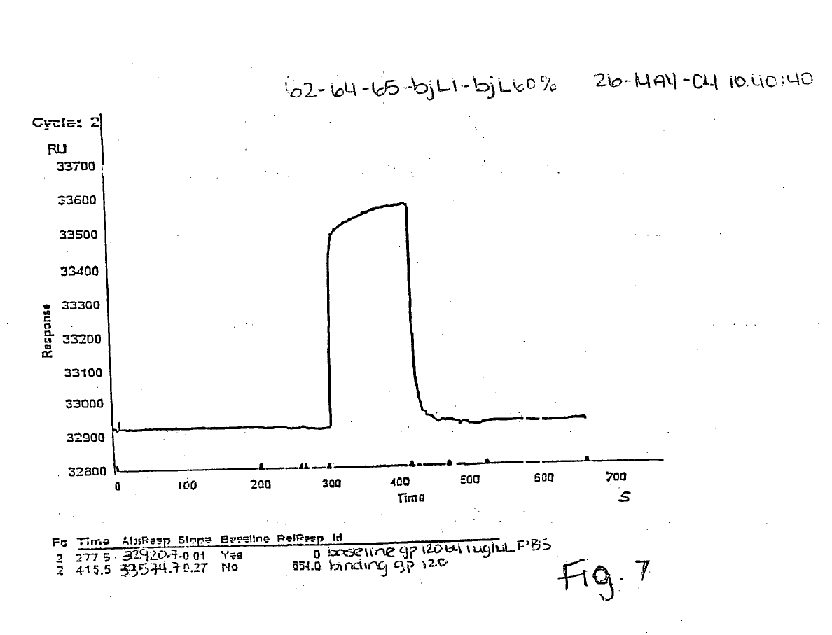

The present study adopted CD and JHR. CD is rubia-one of ingredients of this medicinal herbs composition. The results of conjugation effects of gp41 (transmembrane protein) were shown in FIGS. 6 and 7. The genetically recombined gp41 was placed on the chip of BIACORE analyzer. After adding a certain concentration of rubia or JHR extract, the instrument could detect whether or not there was a conjugation. Both CD and JHR exhibited conjugations with gp41.

### Example 4

### Combination Medication of this Invented Medicinal Herbs Composition and Other AIDS-Treating Drugs

Object: It is to observe if there is any synergistic effect between ZL-1 and AZT. One of component medicines of ZL-1-JHR will be used.

Method: For the experiment, the MAGI test method was adopted (the same as above)

\textcircled{1} Single-Agent Medication:
AZT from 1 uM to 3.9 nM, 5 doses designated AZT1-5
JHR₁₋₅ from 400 ug/ml to 1.56 ug/ml, in 5 doses
IC₅₀ obtained respectively.

\textcircled{2} Combined Application

Half AZT plus half JHR-1 as one sample;

AZT1 plus any of JHR1-JHR5;

AZT2, AZT3, AZT4, AZT5 was combined with any of JHR-1-JHR-5. So a total of 25 concentration combinations can be used. Each concentration was set in duplicate wells (2 wells). Additional group was taken as cell and virus controls.

\textcircled{3} Comparing each drug combination with the virus group to have the inhibition rate. The inhibition rate of each drug combination was compared with the sole AZT IC50 to obtain the difference.

The below Table 2 shows the combined-use results of JHR-1 and AZT.

TABLE 2

| Drug | ED50 | | AZT/A + J |
|---|---|---|---|
| JH | 21.5 | μg/ml | |
| AZT | 46 | nM | |
| AZT-JHR-1 | 5.8 | nM | 7.93 |
| AZT-JHR-2 | 10 | nM | 4.60 |
| AZT-JHR-3 | 16.7 | nM | 2.75 |
| AZT-JHR-4 | 33.5 | nM | 1.37 |
| AZT-JHR-5 | 40.6 | nM | 1.13 |

As shown in Table 2, the medicinal herbs composition has the synergistic drug effects with AZT. The maximal dose group may potentiate by 7.9 folds.

### Example 5

### Inhibiting Effects of One of the Components of Medicinal Herbs Composition of the Present Invention on the HIV Strains Resistant to Protease Inhibitors

The present inventors further studied the inhibiting effects of medicinal herbs composition of the present invention on the HIV strains resistant to protease inhibitors.

HIV-1 virus was the strain resistant to protease inhibitors with virulence at 5.7×10⁴ IU/ml. The Hela-CD₄ cell was adopted. The MAGI test method was used to observe the effects of JHR and see if there was any cross-reactivity. Results indicated that dose of JHR was 0.4 mg/ml and the inhibition rates of virus for 5 μl or 8 μl were both as high as 100%. These demonstrated some effects upon the strains resistant to protease inhibitors. The results refer to Table 3.

TABLE 3

| | Virus load | Drug (0.4 mg/ml) | Inhibition rate % |
|---|---|---|---|
| PRIV* | 5 μl | JH | 100 |
| | 8 μl | JH | 100 |

*PRIV is the viral strain resistant to protease inhibitors.

### Example 6

### Safety Experiment of Medicinal Herbs Composition of the Present Invention

(A) Treatment index detection in vitro of medicinal herbs composition of the present invention refers to Table 4.

TABLE 4

| TC₅₀ (mg/ml) | IC₅₀ (μg/ml) | TC₅₀/IC₅₀ |
|---|---|---|
| 8.34 | 44.3 | 188.3 |

(B) toxicity experiment in vivo of medicinal herbs composition of the present invention:

(1) Results of acute toxicity experiment indicate no toxicity observed for doses over 20 g/kg in intragastric dosing for rats.

(2) Results of sub-acute toxicity experiment indicate after continuous intragastric dosing for 6 months, rats grow normally in the large, medium and small dose groups, ALT, BUN, RBC, WBC with DC are all normal and no abnormality are observed on pathological slides for such organs as heart, liver, kidney, spleen, lung, pancreas, brain, testis and ovary.

### Example 7

### Clinical Trial of the Medicinal Herbs Composition of the Present Invention

Patient Source: In 2002, after treating 1,000 patients at Xincai and Shangqiu counties, Henan province, blood was drawn from 60 patients. At the sample selection, the subjects were those infected with HIV through donating blood to others from 1992 to 1995. There were 19 males and 41 females among a total of 60 persons with ages of 27-58 year-old. The median age was 38.9 year-old and all were farmers. Among them, 59 cases were tested positive for HCV-Ab, 7 cases positive for syphilis PPR test, 2 cases having TB co-infections and all of them are negative for HBV tests.

Administration Method 4 capsules, t.i.d, orally.

1. Changes of Viral Load Before and after Treatment:

The patient blood samples were drawn before treatment and 6 months post-treatment. The plasma was centrifuged and stored at −80° C. At the time of measurement, according to the instructions of Rocher quantitative RNA PCR and reagent kits, at first RNA of the sample was taken and then the viral load (VL) was measured with the Rocher instrument. The results were shown in Table 5.

TABLE 5
ZL-1 post-treatment VL changes

| | Pre- | 6 months | Changes of | | |
|---|---|---|---|---|---|
| Number | treatment | post-treatment | viral load | Evaluation | % |
| 2 | 10^{5.951} | 10^{3.255} | 2.696 | ↓ | |
| 9 | 10^{4.155} | 10^{2.668} | 1.487 | ↓ | 3/10 |
| 59 | 10^{3.599} | 10^{2.668} | 0.931 | ↓ | |
| 1 | 10^{5.935} | 10^{6.143} | 0.208 | → | |
| 14 | 10^{5.520} | 10^{5.560} | 0.04 | → | 4/10 |
| 24 | 10^{5.072} | 10^{4.884} | 0.188 | → | |
| 30 | 10^{4.990} | 10^{5.299} | 0.309 | → | |
| 51 | 10^{4.433} | 10^{5.021} | 0.588 | ↑ | |
| 11 | 10^{2.823} | 10^{3.436} | 0.613 | ↑ | 3/10 |
| 28 | 10^{4.274} | 10^{4.856} | 0.582 | ↑ | |

2. Changes of P₂₄-Ag Before and after Treatment

Use the Microelisa method and reagents to measure the P₂₄-Ag of the above samples. Results shown in Table 6.

TABLE 6
ZL-1 post-treatment HIV P₂₄₋Ag changes

| | | Changes | |
|---|---|---|---|
| | 6 months | of viral | |
| Pre-treatment | post-treatment | load | Evaluation |
| 10^{3.25} | 10^{2.292} | 0.958 | >0.5 ↓ |
| 10^{5.131} | 10^{4.586} | 0.545 | >0.5 ↓ |
| 10^{5.18} | 10^{5.501} | 0.318 | <0.5 → |
| 10^{4.187} | 10^{5.125} | 0.94 | >0.5 ↑ |

As shown in the results in Table 6, after taking the medicinal herbs composition, the VL in patient's plasma dropped considerably after treating for 6 months. 70% of patients had no change while the value slightly rose in 30%.

3. CD₄ Measurement Before and after Treatment:

For the collected blood samples, run the whole blood on flow cytometry to measure the CD₄. Results shown in Table 7.

TABLE 7
CD₄ measurement of 60 patients

| | | Before | After medication | | |
|---|---|---|---|---|---|
| | | medication | 1 month | 3 month | 6 month |
| CD4 | Group 1 (30) | 135.8 | 264.6 | 288.3 | 297.3 |
| | Group 2 (30) | 94.2 | 223.8 | 274.2 | 293.1 |
| | | 115 | 244.2 | 281.3 | 295.2 |
| Total | CD₄ increase % | | 112.3 | 144.6 | 156.7 |

As seen from the results in Table 7, the patient's blood CD₄ levels rose markedly after taking the medicinal herbs composition. The said medicinal herbs composition was shown to able to boost the patient's blood CD₄ levels. The CD₄ count doubled after taking medication for one month. With more medications taken, the CD₄ number shot up continuously. The increase was 156.7% within 6 months. The CD₄ count approached 300. The normal standard of CD₄ is above 200 toward recovery while it is lower than 200 in the patients.

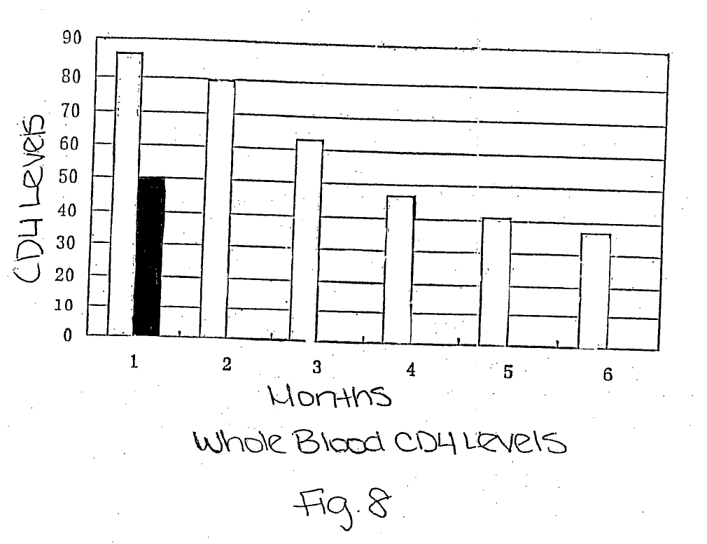

Furthermore, the auxiliary medications currently used for the AIDS patients in mainland China have to be approved on the required rationale of boosting the patient's blood CD₄ levels by over 30% within 50% or more of the trial subjects. The present inventors also measured the whole-blood CD₄ levels after treatment with the said medicinal herbs composition ZL-1 for 6 months. The results were shown in FIG. 8. In FIG. 8, the said medicinal herbs composition boosted the CD₄ level by over 30% in 86% patients, over 50% in 80% patients and even 400 folds in 37% patients.

4. Observation of 60 patients with regards to the changes of clinical symptoms before and after treatment. Results are shown in Table 8A, 8B.

TABLE 8A

| | | | | Flue frequency | | | | | |
|---|---|---|---|---|---|---|---|---|---|
| | Body weight | | | and severity | | | Weakness | | |
| | ↑ | ↓ | → | ↑ | ↓ | → | ↑ | ↓ | → |
| Persons | 21 | 12 | 18 | 12 | 23 | 20 | 23 | 14 | 16 |
| Total | 53 | 53 | 53 | 53 | 53 | 53 | 53 | 53 | 53 |
| No. | | | | | | | | | |
| Persons | | | | | | | | | |
| % | 39.6 | 22.6 | 34 | 22.6 | 43.4 | 37.7 | 43.4 | 26.4 | 30.1 |

| | | |
|---|---|---|
| ↑ & → 73.6% | ↓ & → 81.1% | ↓ & → 56.5% |

→

TABLE 8B

| | Appetite | | | Diarrhea frequency | | | Itches & rashes | | |
|---|---|---|---|---|---|---|---|---|---|
| | ↑ | ↓ | → | ↑ | ↓ | → | Aggravated | Relieved | → |
| Persons | 17 | 14 | 22 | 5 | 9 | 2 | 7 | 10 | 6 |
| Total | 53 | 53 | 53 | 16 | 16 | 16 | 23 | 23 | 23 |
| No. Persons | | | | | | | | | |
| % | 32 | 26.4 | 41.5 | 31.2 | 56.2 | 12.5 | 30.4 | 43.4 | 26 |

| | | |
|---|---|---|
| ↑ & → 73.6% | ↑ & → 68.7% | ↑ & → 69.4% |

The above data demonstrated that the clinical symptoms and signs were improved markedly.

### Example 8

The present example describes a typical case of a 32-year-old person named Ni X X from Liaoning province. Refer to Table 9 for the changes of virus load before and after treatment with the medicinal herbs composition of the present invention. The dosing method was the same as that of Example 1.

TABLE 9
Changes of viral load for one patient receiving treatment of the
medicinal herbs composition

| Date | RNA copy/ml |
|---|---|
| Oct. 30, 1998 (1 w pre-treatment) | >500,000 |
| Nov. 5, 1998 (beginning medication) | >500,000 |
| Nov. 12, 1998 (1 week post-treatment) | >500,000 |
| Nov. 26, 1998 (3 week post-treatment) | 330,000 |
| Apr. 9, 1999 (22 week post-treatment) | 88,960 |
| Apr. 16, 1999 (23 week post-treatment) | 78,771 |

1. A medicinal herbs composition comprising:
10-30 weight parts of extract powders of Scutellaria baicalensis Georgi;
10-25 weight parts of Rubia cordifolia L;
18-25 weight parts of powders of ginsenoside; and
30-55 weight parts of powders of Cordyceps sinensis (Berk.) Sacc., based on 100 weight parts of the composition.

2. The medicinal herbs composition according to claim 1, wherein the medicinal herbs composition comprises 18 weight parts of extract powders of Scutellaria baicalensis Georgi, 17 weight parts of Rubia cordifolia L, 18 weight parts of powders of ginsenoside, and 47 weight parts of powders of Cordyceps sinensis (Berk.) Sacc. based on 100 weight parts of the composition.

3. The medicinal herbs composition according to claim 1, wherein the extract powders of Scutellaria baicalensis Georgi and Rubia cordifolia L or ginsenoside powder are ethanol extracted powders and the powder of Cordyceps sinensis (Berk.) Sacc is a powder of natural Cordyceps sinensis (Berk.) Sacc or artificially cultivated Cordyceps sinensis (Berk.) Sacc.

4. A preparation for the medicinal herbs composition of claim 1, comprising the steps of:
a) Crushing the crude herb of Scutellaria baicalensis Georgi as raw material medicine, adding 4-5 folds of ethanol for immersing for at least 6 hours, pouring the immersed Scutellaria baicalensis Georgi and immersion solution together into an osmotic filtration tank for filtration in rate at 1-4.5 ml/kg.minute⁻¹; then adding ethanol and osmotic-filtrating simultaneously until making the collected osmotic filtration fluid equal in weight to 10-20 folds of the crude herbs; collecting and concentrating the osmotic filtration fluid to form final concentrated solution having weight ratio of water and concentrated fluid at 1:1.1-1.35; spray drying the concentrated fluid into extract powder of Scutellaria baicalensis Georgi.
b) Simultaneously or sequentially crushing the crude herb of Rubia cordifolia L, adding 4-5 folds of ethanol for immersing for at least 6 hours, pouring the immersed Rubia cordifolia L, and immersion solution together into an osmotic filtration tank for filtration in rate at 1-4.5 ml/kg.minute⁻¹, then adding ethanol and osmotic-filtrating simultaneously until making the collected osmotic filtration fluid equal in weight to 10-20 folds of the crude herbs; collecting and concentrating the osmotic filtration fluid to form final concentrated fluid having weight ratio of water and concentrated fluid at 1:1.1-1.35; spray drying the concentrated fluid into extract powder of Rubia cordifolia L.;
c) mixing 10-30 weight parts of extract powder of Scutellaria baicalensis Georgi, 10-25 weight parts of extract powder of Rubia cordifolia L, 18-25 weight parts of ginsenoside powder and 30-55 weight parts of powder of Cordyceps sinensis (Berk) Sacc to obtain the medicinal herbs composition based on 100 weight parts of the composition.

5. The preparation of claim 4, wherein the said ethanol is 30-70% ethanol.

6. The use of the medicinal herbs composition of claim 1 for preparation of anti-HIV drugs.

7. The use of claim 5, wherein said medicinal herbs composition is used in combination with the ordinary anti-HIV drugs.

8. The use of claim 7, wherein the said ordinary anti-HIV drugs includes AZT, DDC, DDI, saquinavir, ritonavir, indinavir sulfate and nefinavir.
